(19) United States
(12) Patent Application Publication
(10) Pub. No.: US 2007/0221362 A1
(43) Pub. Date: Sep. 27, 2007

(54) Disinfection System

(76) Inventors: Murray Kenneth Thomas Stewart, New South Wales (AU); John David Aitken, New South Wales (AU)

Correspondence Address: EDWARDS ANGELL PALMER & DODGE LLP, P.O. BOX 55874, BOSTON, MA, 02205, US

(21) Appl. No.: 11/587,369

(22) PCT Filed: Apr. 22, 2005
(86) PCT No.: PCT/AU05/00584

§ 371 Date: Mar. 16, 2007

Related U.S. Application Data
(60) Provisional application No. 60/565,052, filed on Apr. 24, 2004.

(30) Foreign Application Priority Data
Apr. 23, 2004 (AU) 2004902177
Nov. 18, 2004 (AU) 2004906575

(51) Int. Cl. F28D 7/00; A61L 2/00; F28F 1/00; F25B 27/02

(52) U.S. Cl. 165/57; 210/175; 62/235.1; 62/238.1

(57) ABSTRACT

Apparatus for disinfecting fluid, the apparatus including a preheat heat exchanger (4) and a disinfection tank (5). The preheat exchanger heats the fluid to a first temperature and includes a first inlet for receiving the fluid (2), a first outlet for supplying preheated fluid at the first temperature, a second inlet for receiving disinfected fluid substantially at a second temperature and a second outlet for supplying the disinfected fluid (3). The disinfection tank heats the fluid to a second temperature and includes a heat source (10), an inlet for receiving the preheated fluid, a heat exchanger coupled to the inlet for heating the preheated fluid to a second temperature to thereby disinfect the fluid (11) and, an outlet coupled to the heat exchanger for providing the disinfected fluid to the second inlet of the preheat heat exchanger.

### BACKGROUND OF THE INVENTION

The present invention relates to a method and apparatus for disinfecting fluids, and in particular, to apparatus for disinfecting fluids using heat treatment, as well as to apparatus for providing a combination supply, and in particular for supplying a combination of hot water, air conditioning and disinfected water.

### DESCRIPTION OF THE PRIOR ART

The reference to any prior art in this specification is not, and should not be taken as, an acknowledgement or any form of suggestion that the prior art forms part of the common general knowledge.

It is well known to provide for disinfection of fluids to destroy or inactivate organisms, viruses and pathogens in the fluids, by heating fluids to a predetermined temperature for a predetermined amount of time. Typically when it is desired to disinfect large or small volumes of fluid this is achieved either by heating the fluid in a holding tank or metallic tank

However, current systems for disinfecting fluid tend to be expensive and energy inefficient, as well as having a short lifecycle due to corrosion of the holding tank, making the supply of large volumes of disinfected fluid an expensive process.

Whilst alternatives have been suggested, these techniques are also typically inefficient. For example, in ensuring sufficient sanitation of sewer water, it is typical to use reverse osmosis which uses membranes that are very costly to operate and run while not fully reusing all of the waste water with significant losses in the treatment process.

Furthermore, when providing facilities in remote areas, such as hot water, disinfected water, air conditioning, and the like, efficiency of operation of systems becomes important, primarily in minimising both operating and environmental costs.

A similar issue is encountered with ships ballast water. Ballast water is used to maintain buoyancy and stability for a ship carrying varying amounts of cargo. In order to achieve this, as the ship is loaded or unloaded it is typical to remove or add ballast water to or from the local harbour. When the ship reaches its destination port, and is unloaded, it is again typical to add or remove ballast water from the ballast tanks. In this instance, this allows the destination port to be contaminated with water from the port of origin which thereby provides a mechanism for marine organisms, pathogens and other contaminants to travel from one port to another.

In order to reduce such risks, ships are required to cycle their ballast water at sea by emptying each of the ballast tanks in turn and replenishing the empty tanks with seawater. This is a complex and time consuming process and incurs significant risks to the safety of the vessel. In particular, when a ballast tank is empty this places undue strain on the hold and can lead to hull breaches. In addition to this, whilst the water is being replenished the ship generally suffers from poor stability and can therefore capsize in heavy seas.

As a result of this, ship's captains often are unable to cycle the ballast water as required, often leading to contamination of different harbours.

In remote resorts it is also typical to provide a number of independently operating systems in order to provide for all the facilities requirements. This will typically include a supply of hot water, and an electricity supply. The electricity is used for general lighting and power applications, as well as to drive air conditioning machinery. In addition to this, it may be necessary to provide a degree of water purification, which again typically requires energy hungry apparatus. As a result of this, for a resort capable of catering for 300 guests, it is typical to require at least three generators.

As an alternative to electrically driven air conditioning, it is possible to utilise absorption chillers. Typically however such systems are also inefficient when used independently from other apparatus, and therefore unsuitable for many applications and further to the operating and environmental costs.

### SUMMARY OF THE PRESENT INVENTION

In a first broad form the present invention provides apparatus for disinfecting fluid, the apparatus including:
(a) a preheat heat exchanger for heating the fluid to a first temperature, the preheat heat exchanger including:
   (i) a first inlet for receiving the fluid;
   (ii) a first outlet for supplying preheated fluid at the first temperature;
   (iii) a second inlet for receiving disinfected fluid substantially at a second temperature; and,
   (iv) a second outlet for supplying the disinfected fluid; and,
(b) a disinfection tank for heating the fluid to a first temperature, the disinfection tank including:
   (i) a heat source;
   (ii) an inlet for receiving the preheated fluid;
   (iii) a heat exchanger coupled to the inlet for heating the preheated fluid to a second temperature to thereby disinfect the fluid; and,
   (iv) an outlet coupled to the heat exchanger for providing the disinfected fluid to the second inlet of the preheat heat exchanger.

Typically the heat exchanger has a predetermined length.

The heat exchanger is typically formed from a convoluted pipe, or a coiled pipe. In the case of a coiled pipe, this is preferably adapted to reduce the effects of channelling within the pipe.

The heat source typically includes a primary circuit and at least one of:
(a) a heating element; and,
(b) a second heat exchanger coupled to a source of hot fluid.

The hot fluid is preferably heated by at least one:
(a) waste heat from equipment; and,
(b) solar heating.

The disinfection tank is preferably a reverse acting califorier, in which case it may be a Rotex™ SC500.

In this case, the heat exchanger is preferably a PE-X heat exchanger. The preheat heat exchanger can be a second reverse acting califorier, such as a Rotex™ SC500. in this case, the second inlet and second outlet can be coupled to a primary circuit of the second Rotex™ SC500, with the preheat heat exchanger being a PE-X heat exchanger.

The disinfection tank can include an insulated housing.

The heat source can include a pipe coupled to a boiler.

The fluid at the second temperature may be pressurised.

The fluid can be provided at a predetermined rate, and wherein the heat exchanger is adapted to heat the fluid to the second temperature for a predetermined length of time.

In this case, the typically further includes a control system for controlling the predetermined rate.

The control system may include:
(a) a flow control valve; and,
(b) a controller for controlling the flow control valve.

The apparatus can also further include a temperature sensor which generates signals indicative of the second temperature, and wherein the controller controls the predetermined flow rate in accordance with the signals.

The controller is typically a suitably programmed processing system.

In a second broad form the present invention provides a method of operating apparatus for disinfecting fluid, the apparatus including:
(a) a preheat heat exchanger for heating the fluid to a first temperature, the preheat heat exchanger including:
   (i) a first inlet for receiving the fluid;
   (ii) a first outlet for supplying preheated fluid at the first temperature;
   (iii) a second inlet for receiving disinfected fluid substantially at a second temperature; and,
   (iv) a second outlet for supplying the disinfected fluid; and,
(b) a disinfection tank for heating the fluid to a first temperature, the disinfection tank including:
   (i) a heat source;
   (ii) an inlet for receiving the preheated fluid;
   (iii) a heat exchanger coupled to the inlet for heating the preheated fluid to a second temperature to thereby disinfect the fluid; and,
   (iv) an outlet coupled to the heat exchanger for providing the disinfected fluid to the second inlet of the preheat heat exchanger; and,
   wherein the method includes supplying the fluid to the first inlet at a predetermined rate.

In this case, the method of the second broad form can be performed using the apparatus of the first broad form.

In a third broad form the present invention provides a supply system including:
(a) an absorption chiller for using an external heat source to provide chilled fluid;
(b) a fluid disinfection system for using an external heat source to provide disinfected fluid;
(c) a hot water system for using an external heat source to provide heated fluid; and,
(d) a waste heat recovery system to recover waste heat, the waste heat recovery system acting as an external heat source for at least one of the absorption chiller, the fluid disinfection system and the hot water storage system.

Typically the waste heat recovery system includes a heat exchanger coupled to at least one of:
a) a generator; and,
b) a boiler.

Typically the waste heat recovery system provides heat to a selected one of the absorption
chiller, the fluid disinfection system and the hot water system, the system further including a second waste heat recovery system for:
a) recovering waste heat from the selected one of the absorption chiller, the fluid disinfection system and the hot water system; and
b) providing the waste heat to one of the absorption chiller, the fluid disinfection system and the hot water system.

Typically the absorption chiller includes:
a) an evaporator which uses evaporation of a refrigerant to cool fluid received via an inlet, and provide chilled fluid via an outlet;
b) an absorber for:
i) receiving evaporated refrigerant from the evaporator; and,
ii) causing the evaporated refrigerant to be absorbed by a refrigerant-depleted solution to form a solution;
c) a chiller generator for:
   i) receiving the solution from the absorber;
   ii) evaporating refrigerant from the solution using an external heat source heat to create the refrigerant-depleted solution; and,
   iii) providing the refrigerant-depleted solution to the absorber;
d) a condenser for:
   i) receiving evaporated refrigerant from the chiller generator;
   ii) condensing the evaporating refrigerant and generating waste heat; and,
   iii) providing the refrigerant to the evaporator.

Typically the fluid disinfection system includes:
a) a preheat heat exchanger for heating the fluid to a first temperature, the preheat heat exchanger including:
   i) a first inlet for receiving the fluid;
   ii) a first outlet for supplying preheated fluid at the first temperature;
   iii) a-second inlet for receiving disinfected fluid substantially at a second temperature; and,
   iv) a second outlet for supplying the disinfected fluid; and,
b) a disinfection tank coupled to an external heat source for heating the fluid to a first temperature, the disinfection tank including:
   i) an inlet for receiving the preheated fluid;
   ii) a heat exchanger coupled to the inlet for heating the preheated fluid to a second temperature to thereby disinfect the fluid; and,
   iii) an outlet coupled to the heat exchanger for providing the disinfected fluid to the second inlet of the preheat heat exchanger.

Typically the heat exchanger has a predetermined length.

Typically the heat exchanger is formed from a convoluted or coiled pipe.

Typically at least one of the disinfection tank and the preheat heat exchanger are formed from a reverse acting califorier.

Typically the reverse acting califorier is a Rotex# SC500.

Typically the heat exchanger is a PE-X heat exchanger.

Typically the hot water supply includes a reverse acting califorier.

In a fourth broad form the present invention provides a supply system including:
a) a fluid disinfection system for using an external heat source to provide disinfected fluid;
b) a hot water system for using an external heat source to provide heated fluid; and,
c) a waste heat recovery system to recover waste heat, the waste heat recovery system acting as an external heat source for at the fluid disinfection system and the hot water storage system.

Typically the waste heat recovery system is coupled to a generator for generating an electricity supply.

Typically the apparatus is apparatus according to the third broad form of the invention.

In a fifth broad form the present invention provides apparatus for treating ballast water in a vessel, the apparatus including:
a) a preheat heat exchanger for heating the ballast water to a first temperature, the preheat heat exchanger including:
   i) a first inlet for receiving the ballast water from a ballast tank;
   ii) a first outlet for supplying preheated ballast water at the first temperature;
   iii) a second inlet for receiving pasteurised ballast water substantially at a second temperature; and,
   iv) a second outlet for supplying the pasteurised ballast water to the ballast tank; and,
b) a pasteurisation tank for heating the ballast water to the second temperature, the pasteurisation tank including:
   i) an inlet for receiving the preheated ballast water;
   ii) a heat exchanger coupled to the inlet for heating the preheated ballast water to a second temperature to thereby pasteurise the ballast water; and,
   iii) an outlet coupled to the heat exchanger for providing the pasteurised ballast water to the second inlet of the preheat heat exchanger; and,
c) a heat recovery heat system coupled to engines provided in the vessel, the heat recovery system being adapted to heat the pasteurisation tank, thereby allowing the ballast water to be pasteurised.

Typically the first inlet is coupled to the ballast tank at a first level and the second outlet is coupled to the ballast tank at a second level, the second level being higher than the first level to thereby ensure disinfected water is returned to the ballast tank at a higher level.

Typically the apparatus includes apparatus according to the first broad form of the invention.

In a sixth broad form the present invention provides apparatus for treating ballast water in a vessel, the apparatus including:
a) a heat recovery heat system for recovering heat from at least one of an engine and a boiler; and,
b) a fluid disinfection system for heating the ballast water to a predetermined temperature using the recovered waste heat, to thereby disinfect the ballast water.

Typically the apparatus includes apparatus according to the first broad form of the invention.

### BRIEF DESCRIPTION OF THE DRAWINGS

An example of the present invention will now be described with reference to the accompanying drawings, in which:

FIG. 1 is a schematic overview of apparatus for disinfecting fluid;

FIG. 2A is a schematic diagram of a first specific example of apparatus for disinfecting fluid;

FIG. 2B is a schematic diagram of a second specific example of apparatus for disinfecting fluid;

FIG. 3A is a schematic diagram of a third specific example of apparatus for disinfecting fluid;

FIG. 3B is a schematic diagram of a fourth specific example of apparatus for disinfecting fluid; and,

FIG. 4 is a schematic diagram of a fifth specific example of apparatus for disinfecting fluid.

FIG. 5A and 5B are schematic diagrams of examples of systems incorporating a fluid disinfection system;

FIGS. 6A and 6B are schematic diagrams of examples of hot water supply systems incorporating a fluid disinfection system;

FIG. 7A is a schematic diagram of an example of a fluid disinfection system for disinfecting ballast water;

FIGS. 7B to 7E are schematic diagrams of examples of the fluid disinfection system used in the ballast water disinfection system of FIG. 7A;

FIG. 8 is a schematic diagram of an example of an absorption chiller;

FIG. 9 is a schematic diagram of an example of a hot water storage system;

FIG. 10 is a schematic diagram of a first example of a combination system including a fluid disinfection system, an absorption chiller and a hot water storage system;

FIGS. 11A to 11D are a schematic diagram of examples of alternative combination systems;

FIGS. 12A and 12B are schematic diagrams of a system for using a combination system in a distributed resort; and,

FIG. 13 is a schematic diagram of a second example of a combination system including a fluid disinfection system, an absorption chiller and a hot water storage system.

### DETAILED DESCRIPTION OF THE PREFERRED EMBODIMENTS

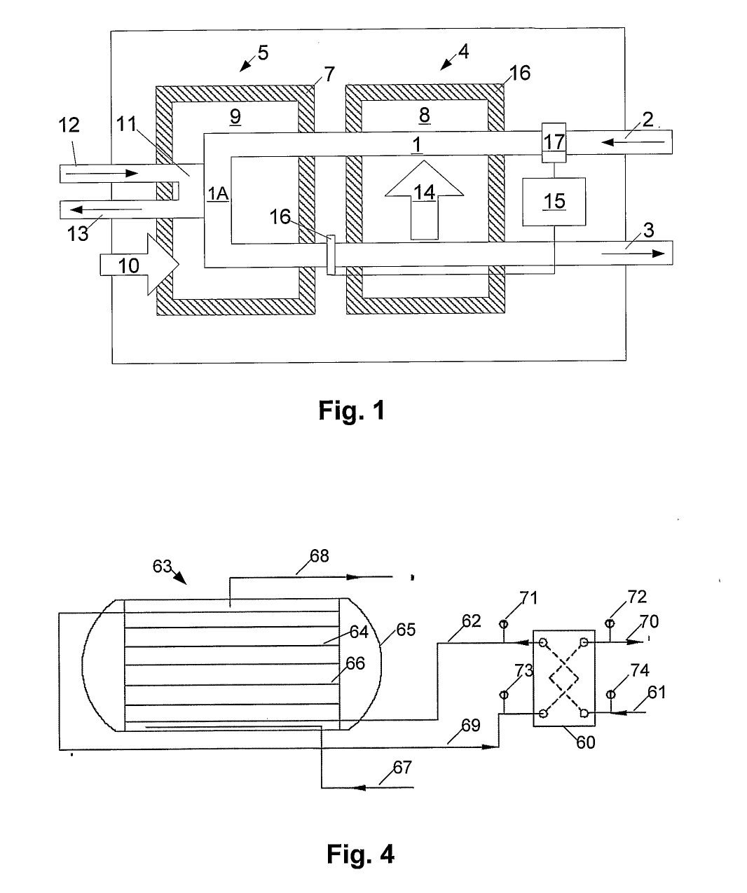

An example of fluid disinfection system will now be described with reference to FIG. 1.

In this example, the fluid disinfection system includes a pipe 1, having an inlet 2 and an outlet 3. The pipe 1 passes through a first heat exchanger 4 and a second heat exchanger 5.

In general, the heat exchangers 4, 5 include respective insulated housings 6, 7 each defining a cavity 8, 9 as shown. The cavity 9 includes a pipe 11 having an inlet 12, and an outlet 13, which is provided adjacent a portion 1A of the pipe 1. In this example, an external heat source 10 is provided to heat water in the cavity 9 to thereby heat the fluid in the pipe 1. In one example this can be achieved by heating another fluid in the pipe 11 additionally, or alternatively other external or internal heat sources 10 may be used to supply heat to the cavity 9, such as electric heating elements or the like. Heating in the first heat exchanger 4 is provided by fluid exiting the second heat exchanger 5, as shown at 14. It will be appreciated however that

Each cavity 8, 9 may be filled with a substance such as water, for retaining heat to thereby improve the efficiency of the heat exchanger as will be appreciated by a person skilled in the art.

In use, fluid to be disinfected is received at the inlet 2 and is transferred along the pipe 1 into the first heat exchanger 4 which provides initial heating of the fluid to a first temperature. The second heat exchanger 5 then heats the fluid to a second temperature. The pipe 1 is arranged so that when the fluid is transferred through the pipe 1 at a predetermined rate, the fluid will spend a predetermined amount of time at the second temperature to thereby ensure the fluid is disinfected.

Fluid exiting the second heat exchanger 5 at the second temperature heats the incoming fluid to the first temperature in the first heat exchanger 4, with the disinfected fluid being provided via the outlet 3. Using waste heat from the fluid exiting the second heat exchanger 5 to pre-heat heat the fluid received at the inlet 2, reduces the amount of heating required in the second heat exchanger 5, by the heat source 10. This allows for a wide range of heat sources to be used, such as waste heat from boilers, generators, air conditioning, or the like as well renewable energy such as solar heating or the like. In the event that insufficient heat is available, any one or more sources can and/or used in conjunction with an internal heating element, or the like.

As will be appreciated by persons skilled in the art, the length of time required to disinfect the fluid will depend on the second temperature used, and the nature of the fluid and contaminants to be deactivated. In general, a higher second temperature will result in the disinfection process taking less time, which in turn allows a higher flow rate of fluid through the pipe 1, for a given pipe length.

To further enhance the volume of fluid which can be disinfected the portion of the pipe within the second heat exchanger 5, shown generally at 1A, is at least partially convoluted to thereby increase the length of the portion 1A within the cavity 9. In order to ensure that the fluid is correctly disinfected it is necessary to control the flow rate in accordance with both the length of the pipe 1, and the nature of the fluid. This may be achieved by providing one or more flow control valves 17, such as proportional flow valves coupled to an appropriate controller 15, to thereby ensure the fluid is maintained at the second temperature-for a sufficient time period. This may be achieved in accordance with signals received, by the controller 15, from a temperature sensor 16.

The controller 15 may be any form of controller that is adapted to respond to signals from the temperature sensor 16, and thereby control the relative opening of the flow valve 17, to thereby maintain a desired flow rate. In one example, this can be achieved using a suitable thermostat and relay. Alternatively however this may therefore be achieved using a suitably programmed processing system, such as a computer, laptop, palm top, PDA, specialised hardware, programmable logic, or the like. This is performed in order to ensure that the fluid receives the required degree of heating to fully disinfect the fluid and destroy any contaminants or the like therein.

Alternatively, the system can be configured to use a predetermined flow rate, which can be defined for example by a fixed orifice. In this case, for example, the inlet 2 may have a fixed cross-sectional area, such that fluid flowing into the pipe 1 flows at a predetermined controlled rate. In this case, it will be appreciated that no form of additional dynamic flow control, such as the provision of the controller 15 is required.

Typically, in the case of the fluid being water, it is necessary for the second temperature to be above 50° C. and preferably above 80° C. In a preferred example, water is heated to a temperature of between 85° C. and 90° C. in the second heat exchanger 5. In this case, the first heat exchanger 4 will typically preheat the water to within a few degrees of the second temperature, and accordingly the first temperature will be in the region of 80 to 85° C.

It will be appreciated however that the temperature used will depend on the application for which the system is used. Thus, for example, if the system is used to treat ballast water, a lower temperature, such as 50° C. may be used, whereas treating fluid for quarantine purposes may require up to 121° C. This will depend factors such as the contaminants to be treated, the intended use of the fluid, any restrictions on time available of disinfection and the degree of external heating available.

In any event, in order to ensure that the fluid is correctly disinfected, to thereby destroy any contaminants, it is necessary to ensure that the fluid is held at the second temperature for a predetermined amount of time, which is based on the flow rate and the length of the pipe portion 1A. Thus, in the example set out above, the pipe 1 is convoluted to increase the length of the portion 1A, to thereby increase the length of time that the fluid remains at the second temperature for at predetermined flow rate.

However, when determining the amount of time the fluid spends in the pipe portion 1A, it is important to take into account the effects of channelling within the pipe 1. In particular, when fluid flows through a pipe, the fluid forms a boundary layer on the inner surface of the pipe. This boundary layer tends to be subject to greater frictional forces than fluid towards the centre of the pipe, and will therefore flow at a slower rate.

As a result, when determining the flow rate and the pipe length it is necessary to ensure that all the fluid remains at the second temperature for the predetermined amount of time. This can be accounted for in two main ways.

Firstly, the theoretical predetermined time for performing disinfection at the operating temperature is determined. The flow rate is then controlled to provide a safety margin, to thereby ensure that the fluid takes longer than the predetermined time to travel through the pipe portion 1A.

Secondly, the pipe portion 1A is designed so as to reduce the effects of channelling. In one example, this is achieved by arranging for the pipe portion 1A to be coiled. The use of a coiled arrangement tends to introduce turbulence and vortices into the flow within the pipe 1, which in turn disrupts the boundary layer, and therefore reduces the effects of channelling. As a result, there fluid flowing through the pipe portion 1A tends to flow at a more uniform rate, thereby ensuring that all the fluid spends an equal amount of time within the pipe portion 1A at the second temperature.

In order to reduce the amount of heating required in the second heat exchanger 5, it will be appreciated by persons skilled in the art, that it is preferable to minimise the difference between the first and second temperatures. Accordingly, it is typical that the first temperature is within a few degrees of the second temperature, and preferable that the first temperature is within one or two degrees of the second temperature.

However, in some circumstances the heat source 10 will provide a fixed degree of heating which heats the fluid in the second heat exchanger by more than a few degrees. This may occur for example if the heat source 10 is formed from waste heat from equipment, and it is required to remove a predetermined amount of heat from the equipment to prevent overheating. In this case, it may be desirable to increase the temperature difference between the first and second temperatures, to thereby ensure that the waste heat is removed.

It will be appreciated by persons skilled in the art that the relative values of the first and second temperatures will depend to a large extent on the configuration of the first heat exchanger 4, and this will therefore be selected depending on the heat source 10.

In the above example, and though the following examples, water or other fluid flow through the pipes or other flow paths is achieved using appropriate pumps which are not shown for clarity purposes, as will be appreciated by persons skilled in the art.

### SPECIFIC EXAMPLES

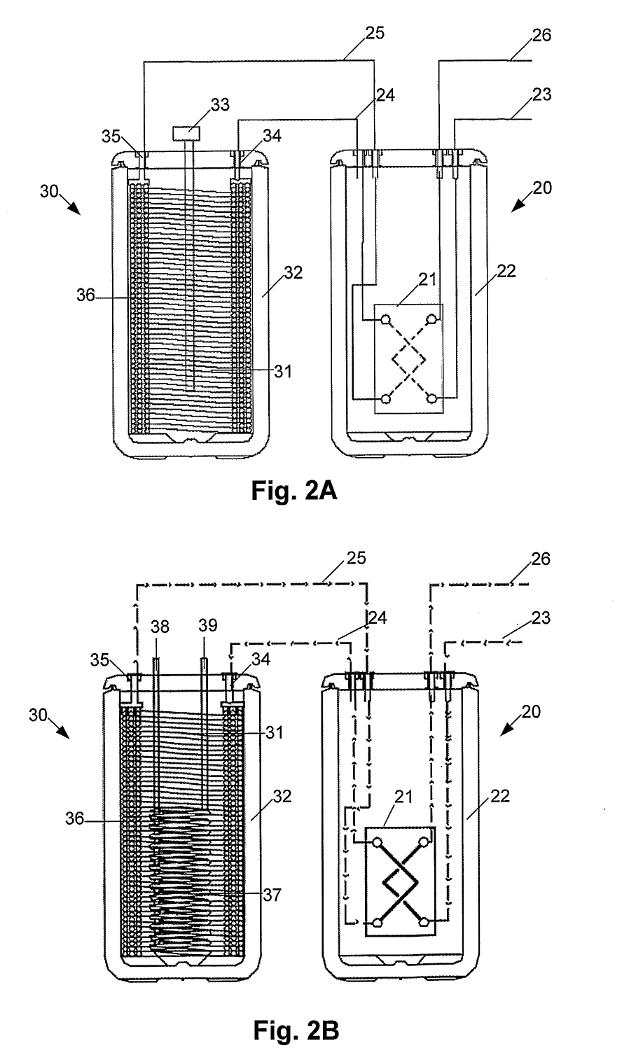

Examples of specific apparatus configurations for performing this will now be described with reference to FIGS. 2 to 4.

In particular, FIG. 2A shows a first configuration including a preheat tank 20, formed from a heat exchanger 21 provided in an insulated housing 22. The preheat tank 20 includes an inlet pipe 23 for receiving the fluid to be disinfected and an outlet pipe 24, for providing fluid at the first temperature. A second inlet pipe 25 is provided for receiving the disinfected fluid at the second temperature, with a second outlet pipe 26 being used to provide the disinfected fluid.

The outlet pipe 24, and the second inlet pipe 25, are connected to a disinfection tank 30 formed from a reverse acting calorifier. This may be formed from any suitable apparatus and generally includes an insulated housing for retaining heat, and a heat exchanger. In one example this is formed from a Rotex™ SC500 heat exchanger, although other apparatus may be used.

The Rotex SC500 heat exchanger or equivalent, which is also referred to as a “Rotex Sanicube”, includes a primary fluid circuit 31 provided in an insulated housing 32. The primary fluid circuit 31 is heated by a heat source 33, to thereby store heat energy. The heat source 33 can be an electric element providing between 2.4 to 24 kilowatts of heating. In this example, a six kilowatt Incalloy 800 element or equivalent, is used to provide the necessary degree of heating.

The disinfection tank 30 includes an inlet 34, and an outlet 35, coupled to a heat exchanger 36, which in this example is a PE-X type heat exchanger, but may an equivalent heat exchanger. In this example inlet and outlet 34, 35 are coupled to the outlet pipe 24, and the second inlet pipe 25, of the preheat tank 20, respectively.

In use, fluid is supplied to the preheat tank 20, and is preheated to the first temperature, which is typically within a few degrees of the second temperature, by the fluid exiting the disinfection tank 30. The preheated fluid is supplied to the inlet 34, and passes through the PE-X heat exchanger 36, where it is heated by one or more of the heating element 33 and the primary fluid circuit 31, to the second temperature, which is at least 60° C., and more preferably at least 85° C.

In this particular configuration and given the limitations of the dimensions and length of the PE-X Heat Exchanger 36, this allows up to 2000 litres of fluid per hour to be disinfected in a typical modular package form. At this rate, fluid typically spends around 4 minutes at the second temperature, which represents more than enough time to disinfect the fluid. In practice at temperatures of between 85° C. and 90° C., disinfection would typically take about 1 minute, and accordingly, even providing a 50% safety margin to ensure disinfection of all fluid, this would allow higher flow rates than 2000 litres per hour to be used in practice.

A second example configuration is shown in FIG. 2B. In this example the electric heat element 33 is replaced by a heat exchanger 37. In particular, the heat exchanger 37 includes an inlet 38 and an outlet 39 to provide fluid heated by an external source, such as solar heating or the like. The heated fluid in the heat exchanger 37 operates to heat the fluid in the primary fluid circuit 31. Apart from this the operation is substantially as described above.

It will be appreciated by persons skilled in the art that this allows waste heat from sources such as air conditioning or the like, or renewable sources such as solar heating, to be used as a suitable heat source for disinfection. This allows disinfection to be achieved using renewable energy sources, or the like.

It will be appreciated that in the event that insufficient heating can be provided via the heat exchanger 37, for example if the temperature of the fluid received at the inlet 38 is too low, then additional heating may be provided using a heating element, such as the heating element 33 described in FIG. 2A above.

This configuration of operation is extremely efficient. In particular, whilst the Rotex SC500 is extremely efficient, it would still require a relatively large amount of energy to heat the disinfecting fluid directly to a suitable second temperature without pre heating. However by preheating the fluid to be disinfected using the fluid leaving the Rotex SC500, this makes the system far more energy efficient thereby vastly improving efficiency.

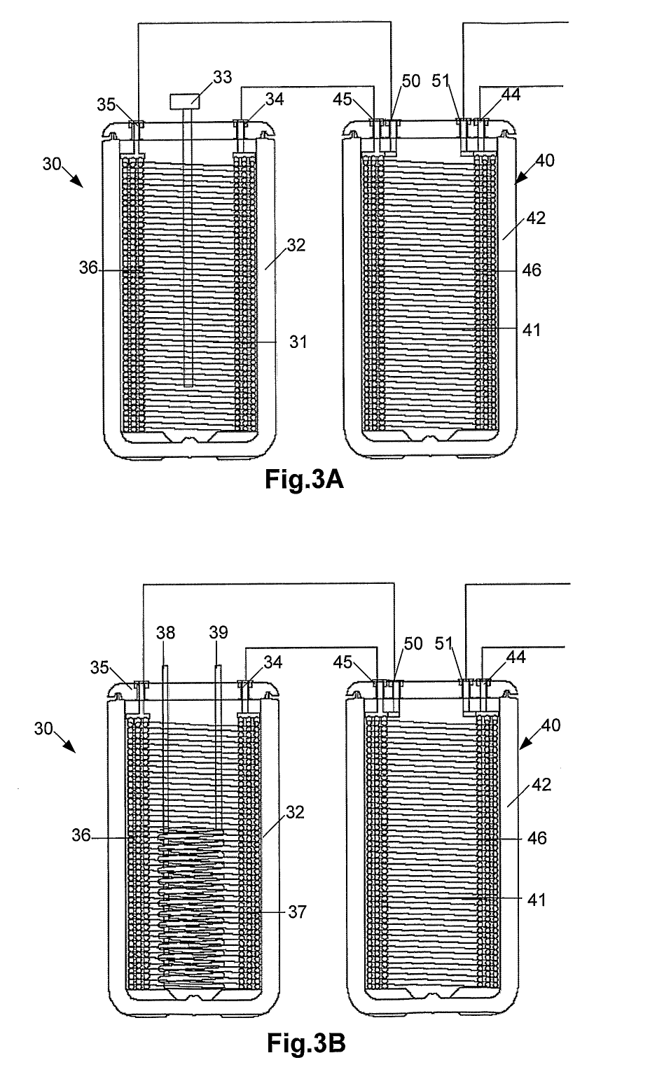

A third example configuration will now be described with reference to FIG. 3A.

In this example, the apparatus is substantially as described above with preheat tank 20 replaced by a Rotex SC500 Tube in Tube device 40. The Rotex SC500 Tube in Tube device 40 is substantially similar to the Rotex SC500 30 described above, with similar reference numerals increased by a value of 10 being used to denote similar integers. However, in this example, the primary fluid circuit 41 is coupled to respective inlet and outlet pipes 50, 51.

In use, the fluid to be disinfected is received via the inlet 44 and passes through the PE-X heat exchanger 46 before being transferred via the outlet 45 to the disinfection tank 30. The fluid is disinfected as described with respect to FIG. 2A, before being provided via the outlet 35 to the inlet 50 of the primary fluid circuit 41. The disinfected fluid flows through the primary fluid circuit 41, and provides preheating of the incoming fluid in the PE-X heat exchanger 46.

It will be appreciated that this configuration is most efficient as the primary fluid circuit generally holds the fluid to be used in heating. However, an alternative configuration could be used in which the fluid to be disinfected is transferred through the primary fluid circuit, with the heated fluid exiting the disinfection tank 30 being provided in the Tube in Tube Pre-heat exchanger 46, or an equivalent.

In this configuration the heat source in the disinfection tank is formed from an Incalloy 800 heating element 32, or equivalent, as described above.

A fourth example configuration is set out in FIG. 3B in which the electric heating element 32 is replaced in with the heat exchanger 37, described above with respect to FIG. 2B.

It will therefore be appreciated that the examples shown in FIGS. 3A and 3B are similar to those shown in FIGS. 2A and 2B and therefore can typically disinfect similar amounts of fluid in similar time frames. Furthermore, whilst the capital cost of Rotex or equivalent,

device, is more expensive than the heat exchanger arrangement shown in FIGS. 2A, 2B, this increased cost is offset by more efficient preheating of the fluid to be disinfected. This therefore reduces the heating requirements in the disinfection tank 30, thereby reducing running costs.

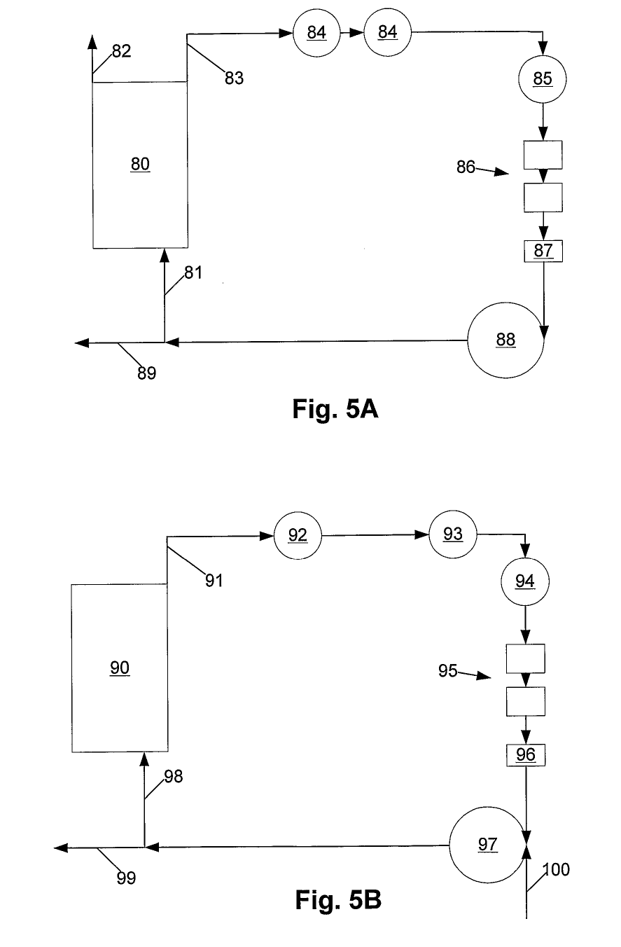

Whilst the above two configurations are both capable of delivering 2000 litres of disinfected fluid per hour in modular form, if it is required to disinfect a higher volume of fluid, such as up to 20,000 litres per hour based on cost, an alternative configuration is required, as will now be described with reference to FIG. 4.

In this example, the apparatus is formed from a heat exchanger or equivalent, 60 having a first inlet 61 for receiving the fluid to be disinfected and an outlet 62 for transferring the preheated fluid to a reverse acting calorifier 63, which is either pressurised or unpressurised. The reverse action calorifier includes a pipe 64 provided in a tank 65. The pipe 64 forms a heat exchanger which is heated by heat source in the form of a pipe 66, which is coupled to a heat source via an inlet 67, and an outlet 68. Disinfected fluid is transferred via a second inlet 69, through the heat exchanger 60 to a second outlet 70.

In a preferred example, the pipe 64 is coiled as described above to reduce channelling effects in the system. However, as an alternative the flow rates of the fluids can be controlled to thereby provide a suitable safety margin to ensure suitable disinfection.

In this example, by providing heating using a heat source, which may be either based on waste heat from equipment, such as air conditioning, or the like, or direct heating, for example from a boiler, this again allows the fluid to be disinfected to be raised to a second predetermined temperature. In this example, the second predetermined temperature can be increased as compared to the second temperature provided in the first through fourth configurations to allow faster disinfection to be achieved.

To prevent the fluid being disinfected from boiling, the system can be pressurised, such that the fluid is provided under pressure within the pipe 64.

In this example, temperature gauges 71, 72, 73, 74, may also be provided to monitor the relative first and second temperatures, to thereby ensure that the fluid is correctly disinfected. Thus, for example, as described above with respect to FIG. 1, the flow rate of the fluid may be controlled in accordance with the temperature to thereby ensure that successful disinfection is performed.

It will be appreciated that the above described system is suitable for disinfecting a wide range of fluids in high volume. In particular, the system is ideally suited for disinfecting both grey fluid, which includes fluid from showers, wash basins, dishwashers and washing machines, and black fluid, which includes fluid from toilets, septic systems etc, and which typically contains amounts of contaminants, such as Faecal coliforms and bacteria, as well target species of organisms, bacteria, pathogens and compounds, such as oestrogen, nitrates, phosphates, pharmaceuticals or the like.

The contaminants which can be deactivated will depend on the disinfection conditions, such as the temperature and time used.

Due to the improved quality of disinfected fluid compared to other fluid cleaning techniques it is possible to utilise the system in a greater variety of circumstances. Thus, for example, the systems can be used to treat effluent from sewage plants and septic tanks, as well as waste water from industry, making it safe for re-use or disposal. In addition to this, animal effluent can be disinfected for use in irrigation of crops or pasture. Polluted river or dam water can be disinfected allowing it to be used as potable water. The system can also be used to disinfect ballast water on ships or boats, allowing the ballast water to be safely returned to the sea or rivers.

When the disinfection system is used to treat black fluids, such as sewer water, it is typical to provide preliminary processing of the fluid prior to disinfection. In particular, the black fluid may be provided to a bio digester, including bio filters such as Zabel A300 bio filters. The fluid will then typically undergo further filtering to remove larger pieces of debris, before being disinfected using one of the above described systems, as will be appreciated by persons skilled in the art.

The systems are also typically cost effective to build and maintain.

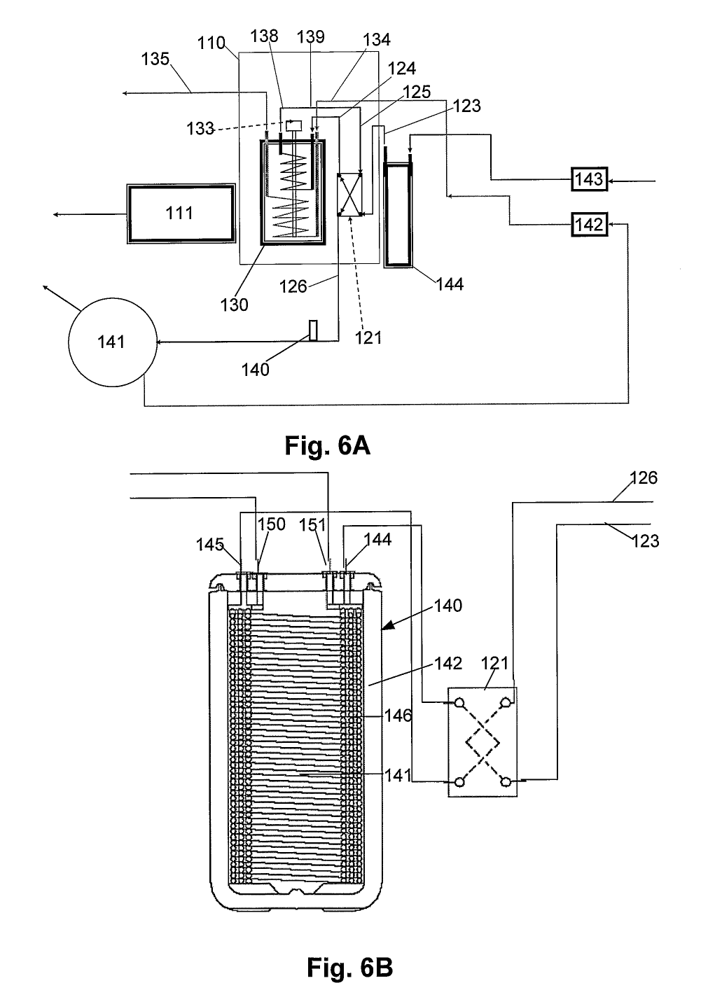

An example for a system for providing disinfected water to a residential unit block 80 will now be described with reference to FIG. 5A. In particular, as shown the residential unit block 80 has a recycle water inlet 81 a black water outlet 82 and a grey water outlet 83. The grey water outlet 83 is coupled, via two surge tanks 84, to a sand filter or biological digester (such as sceptic tank, aerated tank, an aerated waste water treatment system (AWTS), activated sludge plant) 85 and a fluid disinfection system 86. The fluid disinfection system 86 is similar to those described above with respect to FIGS. 1 to 4. The fluid disinfection system 86 is coupled, via a low dose chlorine pump 87, to a recycle water storage tank 88. An output of the recycled water storage tank 88 then supplies water via the recycle water inlet 81 to the residential unit 80 or via an irrigation pipe 89 to an environment for irrigation.

In use, grey water obtained from the unit block 80 is therefore passed through the surge tanks 84 to allow the grey water to be aerated and then filtered using the back flush sand filter 85. This removes any macro sized contaminants. The water is then disinfected using the fluid disinfection system 86, with residual treatment being provided by a low dose chlorine pump 87 as shown. This is used to reduce post-disinfection contamination. The recycled water can then be stored in the storage tank until it is required to be supplied to the unit block 80 or via the irrigation line 89 to an area for irrigating.

For a typical unit block of 150 units there would typically be approximately 412 bedrooms which in turn would result in the production of 123,000 litres of waste water per day. Of this, approximately 83,000 litres would be grey water and 40,000 litres black water.

Accordingly, the grey water can be disinfected using a fluid disinfection system 86, which is capable of disinfecting approximately 3,500 litres per hour.

This provides sufficient water to allow water to be used as follows:
laundry reuse 23 percent of total water per day which equals 28,000 litres per day;
toilet reuse 32 of total water percent per day which equals 40,000 litres per day; and,
irrigation reuse 15,000 litres per day.

The remaining 45 percent total water per day, which is used for hand basins, bathing and kitchen purposes is typically provided via mains water supplies.

A second example of such a system is shown in FIG. 5B in which a set of dwellings 90 are coupled via a waste outlet 91 to an aerated waste water treatment plan 92, an auto back-flush sand filter 93, a surge tank 94 and then to a fluid disinfection system 95. The fluid disinfection system provides disinfected water to a chlorine pump 96 and a recycle water storage tank 97. The storage tank 97 provides recycle water to the dwellings via an inlet 98 or via an irrigation line 99 for irrigation purposes. Additional top-up water may be supplied via a top-up line 100.

Thus it will be appreciated that this functions in a manner similar to that described above with respect to FIG. 5A.

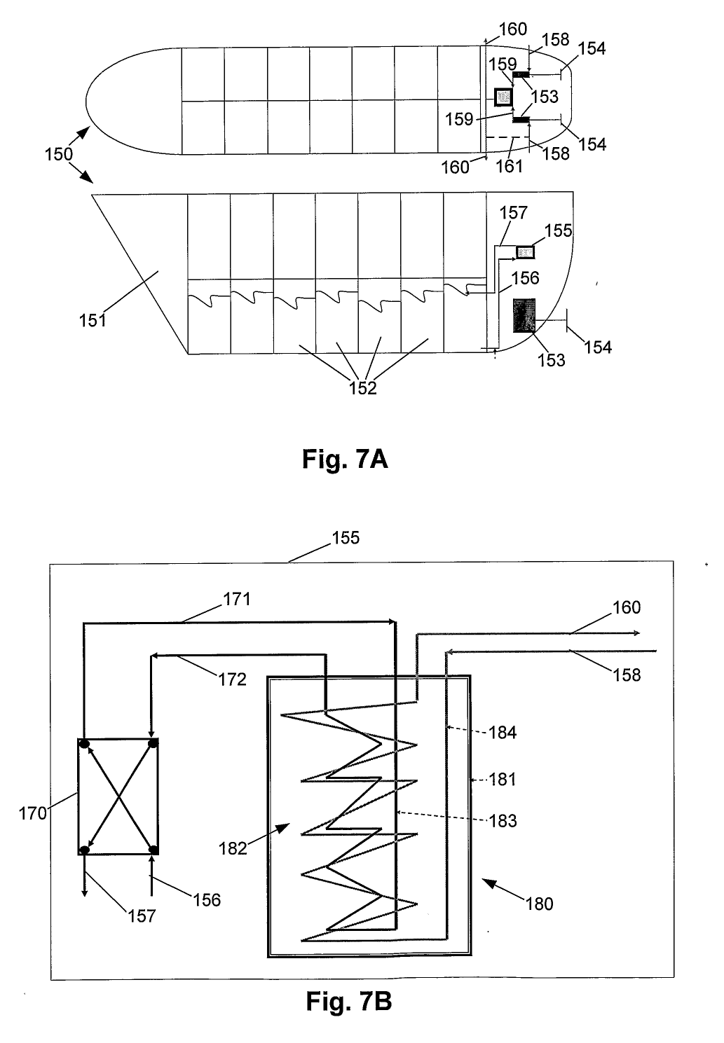

An example of a system for providing fluid disinfection and hot water together with electricity will now be described with reference to FIG. 6A.

In this example, a fluid disinfection system 110 is provided, which similar to that shown in FIG. 2A with reference numerals increased by 100 for like elements. In this particular instance the heat exchanger 130 forms part of the hot water system as well as being part of the fluid disinfection system and therefore includes an additional heat exchange coil 137. This may be similar to the heat exchange coil 37 shown in FIG. 2B or the tube in tube coil 46 shown in FIG. 3A, as shown in more detail in FIG. 6B.

The fluid disinfection system 110 is coupled via the outlet 126 to a chlorine pump 140 and then to a storage tank 141. The storage tank 141 is coupled via a storage pump 142 to the inlet 134 to allow water to be heated, which in turn provides hot water for showers via the outlet 139. In addition to this, a pump 143 is provided to supply water via a back-flush carbine filter 144 to the inlet 123.

In this example, a generator 111 is provided to supply additional electricity for lighting and the like. Waste heat from this generator may be utilised in heating the heat exchanger 130, for example through the use of a heat recovery system which heats water and cycles through the cavity of the heat exchanger 130, as well as to power the heating element 133 if additional heating is required.

This form of system can be used for example in remote areas, or cir areas with effected electricity supplies. This makes the system especially suitable for use in relief areas following disasters or the like where it is often necessary to establish electricity and water supplies rabidly. In particular, a single device weighing less than one ton can supply 20 tons of water per day, thereby vastly reducing transport burdens on the relief effort.

FIG. 6B shows an example of a fluid disinfection using a heat exchanger 121 similar to that shown in FIG. 2A, and the Rotex vessel 140 similar to that shown in FIG. 3A. This therefore shows the use of the tube in tube heat exchanger, and this can therefore provide a combined be used in the system described above in FIG. 6A.

In this case, rather than provide the heat exchanger in a housing similar to the housing 22 in FIG. 2A a smaller heat exchanger, such as a Swep International compact brazed heat exchanger. This reduces the size and weight of the apparatus and allows the heat exchanger 21; 121 to be mounted to the housing 30; 140.

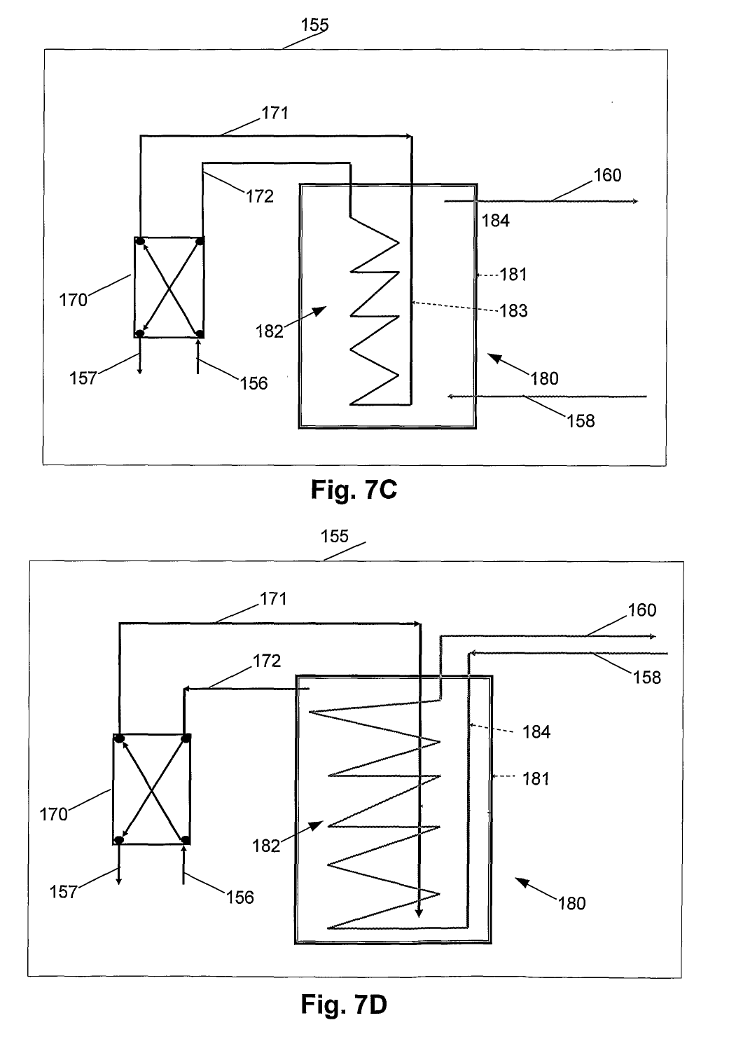

A further example of a use of the fluid disinfection system is shown in FIG. 7A. In particular, this involves operating to disinfect ballast water within ships.

In this example, the ship 150 includes a hull 151 having a number of ballast tanks 152 interconnected via flow-paths or pipes extending between bulkheads 152A. This allows flow of water between the respective ballast tanks 152 to provide for equalisation of ballast water in the tanks. The boat 150 includes an engine 153 for driving propellers 154.

A fluid disinfection system 155 similar to the fluid disinfection systems described above with respect to FIGS. 1 to 4. The fluid disinfection system 155 is coupled to the ballast water tanks 152 via an inlet pipe 156 and an outlet pipe 157. A pump (not shown) is also provided at allow water from the ballast tanks 152 to be pumped through the fluid disinfection system 155.

The engine 153 includes a cooling water inlet 158 which supplies water to a heat exchanger (not shown) provided in thermal contact with the engine. The heat exchanger is coupled via a connecting line 159 to the fluid disinfection system 155, to act as a heat source as shown by the arrow 10 for example in FIG. 1. This may be achieved for example by connecting the connection line 159 to the input 38 of the Rotex heat exchanger 30 shown in FIG. 2B. The outlet 39 is then coupled to an engine water cooling outlet 160 to allow the water to be emitted from the ship

The inlet pipe 156 is coupled to the bottom of the fluid disinfection tanks 152 whilst the outlet pipe 157 to the top of the ballast water tanks 152. Thus, water is removed from the bottom of the ballast water tanks 152 and returned to the top of the ballast water tanks. The returned disinfected water is generally at a higher temperature than the water in the ballast tanks and will tend to remain near the surface of the ballast water tanks causing stratification of the ballast water due to convention processes. This ensures that the water circulates through the ballast tanks before being disinfected again, thereby ensuring that the water if all disinfected adequately.

In this instance, as the level of heat generated by the engine 152 is typically high, this ensures that disinfection can be achieved using no, or only minimal, additional heating. As a result this provides an efficient mechanism for disinfecting and ballast water thereby allowing it to be returned to the sea. Furthermore, as the system includes few moving parts little maintenance is required making the system suitable for long term use.

In the example shown in FIG. 7A the engines 153 are cooled by water received by the cooling water inlet 158 from the ocean with the fluid being returned to the ocean via the cooling water outlet 160. However, as an alternative, thee engine cooling system may be in the form of a closed system in which water is recirculated around the loop as shown by the dotted line 161 which interconnects the cooling water inlet 158 and the cooling water outlet 160.

A further issue is that the ballast water system can utilise a number of different fluid disinfection system configurations. Examples of these are shown in FIGS. 7B, 7C and 7D.

In FIG. 7B, the fluid disinfection system 155 is formed from a pre-heat heat exchanger 170, such as a plate heat exchanger similar to those described above, and a disinfection heat exchanger 180. The pre-heat heat exchanger 170 is coupled to the ballast water inlet and outlet 156, 157, and to the disinfection heat exchanger 180 via the pipes 171, 172. The disinfection heat exchanger 180 includes a housing 181, with a water filled cavity 182. The disinfection heat exchanger 180 includes coils 183, 184 which are coupled to the cooling water inlet and outlet 158, 160, and to the pipes 171, 172, as shown. Operation is therefore similar to the heat exchangers described above.

However, by use of appropriate coils 183, 184 and interconnecting pipes, this allows both the water being disinfected and the cooling water to be pressurised to allow a significantly higher disinfection temperature to be used.

In the configuration shown in FIG. 7C the engine cooling water is supplied via the pipe 158 directly to the cavity 182 to flow therethrough with the disinfected water being kept in the coil 183. In this case, the heated water from the engine is supplied to the lower portion of the cavity 182 allowing it to rise under convection processes thereby ensuring an even distribution of heat.

In the configuration shown in FIG. 7D the situation is reversed with the disinfected water being supplied directly to the cavity 182 via the pipe 171 to flow therethrough with the engine cooling water being supplied in the coil 184. In this case, the water to be disinfected is supplied to the lower portion of the cavity 182 requiring it to be heated so it rises under convection processes ensuring adequate heating and hence disinfection.

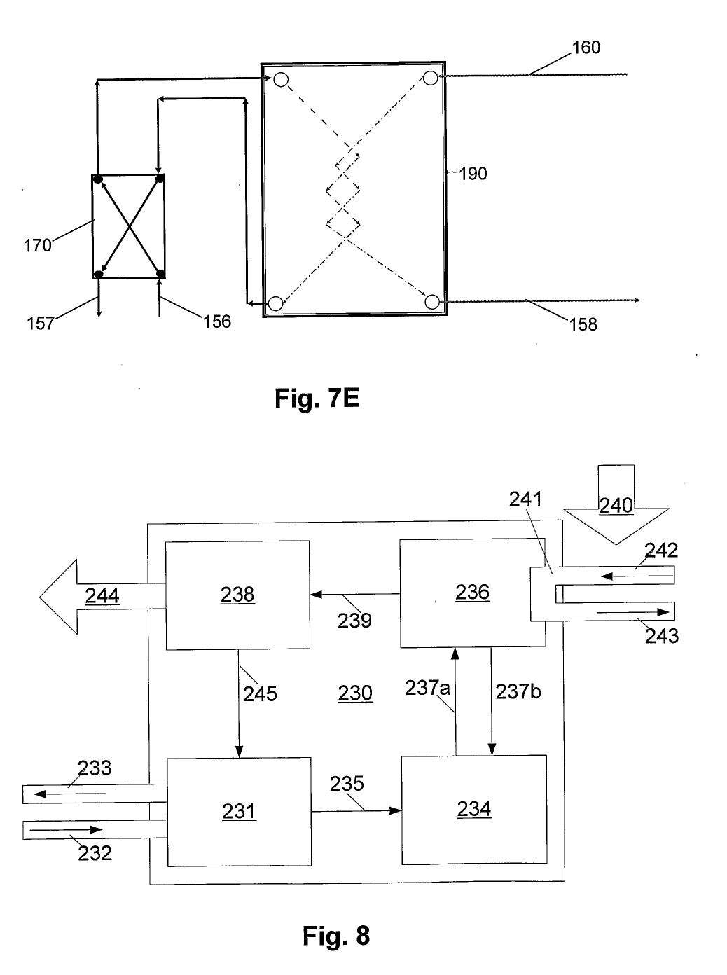

A further example is shown in FIG. 7E in which the disinfection heat exchanger is in the form of a plate and frame heat exchanger 190 as shown. The configuration of the plate and frame heat exchanger can be altered by adjusting the number of plates used, and it will therefore be appreciated that the size of the disinfection heat exchanger will be selected based on the intended application. An example of an absorption chiller will now be described with reference to FIG. 8.

In particular, the absorption chiller 230 includes an evaporator 231 having an inlet 232 and an outlet 233. The evaporator 231 is coupled to an absorber 234, via a pipe 235, which is in turn connected to a generator 236 via pipes 237A, 237B as shown. A pipe 241, having an inlet 242, and an outlet 243 receives heat from an appropriate heat source, as shown at 240, and transfers this to the generator 236. The generator 236 is connected to a condenser 238 via a pipe 239. The condenser 238 typically generates waste heat as shown at 244 and is also coupled to the evaporator 231 via a pipe 245.

The system utilises a solution formed form a combination of a refrigerant and an absorber in order to provide heat transfer mechanisms, as will now be described. Typically the solution is either a water/lithium bromide or an ammonia/water combination as will be appreciated by a person skilled in the art.

In use, the evaporator 231 operates to receive liquid refrigerant from the condenser 238, via the pipe 245. The refrigerant is provided into a low-pressure environment within the evaporator 231, and evaporates, thereby extracting heat from fluid supplied to the inlet 232, via an appropriate heat exchanger. The chilled fluid is then output via the outlet 233, whilst the evaporated refrigerant is transferred via the pipe 235 to the absorber 234, where it is absorbed by a refrigerant-depleted solution.

The solution is transferred via the pipe 237A to the generator 236, which operates to heat the solution using fluid in the pipe 241, thereby causing the refrigerant to be evaporated. The remaining refrigerant-depleted solution returns to the absorber 234 via the pipe 237B, whilst the vaporised refrigerant is transferred via the pipe 239 to the condenser 238. The vaporised refrigerant is allowed to condense with waste heat being output at 244 before being transferred via the pipe 245 to the evaporator 231, thereby allowing the cycle to be repeated.

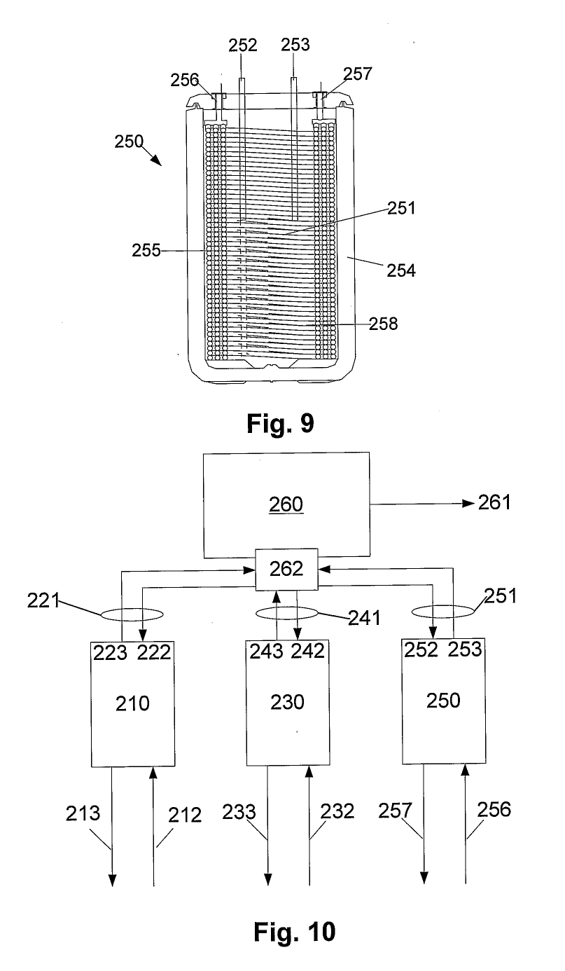

An example of a hot water storage system will now be described with respect to FIG. 9. In particular, FIG. 9 shows an example of a Rotex SC500 250 (also referred to as a “Rotex Sanicube”). The Rotex 250 includes a primary fluid circuit 251, having an inlet 252 and an outlet 253, provided in an insulated housing 254. A PE-X type heat exchanger 255 is also provided, having an inlet 256 and an outlet 257.

In use, water, or another suitable fluid in the primary fluid circuit 251 is heated by an external heat source, and in turn used to heat fluid provided within the insulated 254, as shown at 258. This in turn heats fluid provided in the PE-X heat exchanger 255, which can therefore provide a source of hot water.

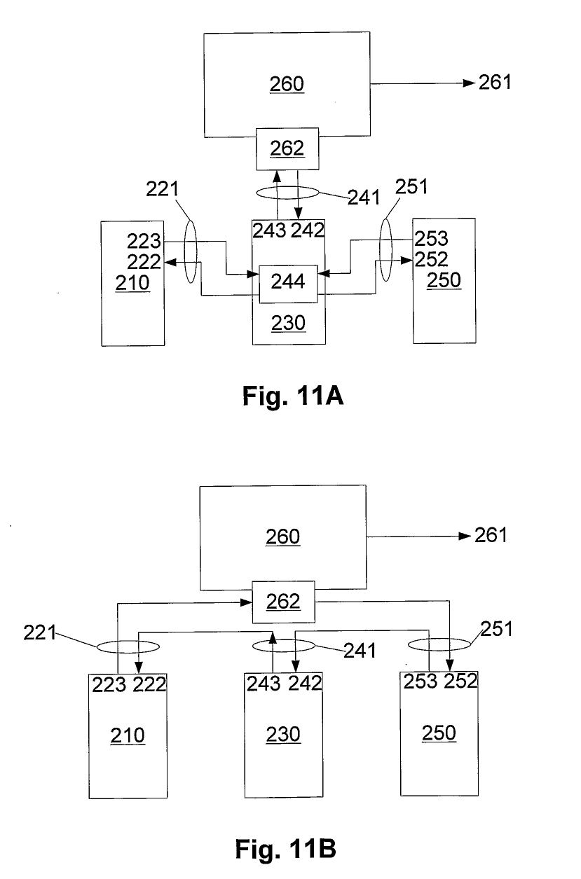

An example of the combination system will now be described with reference to FIG. 10. In particular, as shown at FIG. 10 the system utilises a generator 260 which is coupled to a fluid disinfection system 210, an absorption chiller 230 and a hot water storage system 250 as shown.

In particular, the generator 260 will operate to generate electricity, which is provided via an output 261 as shown. The generator 260 is typically a combustion engine based system, or the like, which therefore generates a significant amount of waste heat. The waste heat is extracted via use of a heat exchanger 262, thereby allowing heat to be provided to the fluid disinfection system 210, the absorption chiller 230 and the water storage system 250.

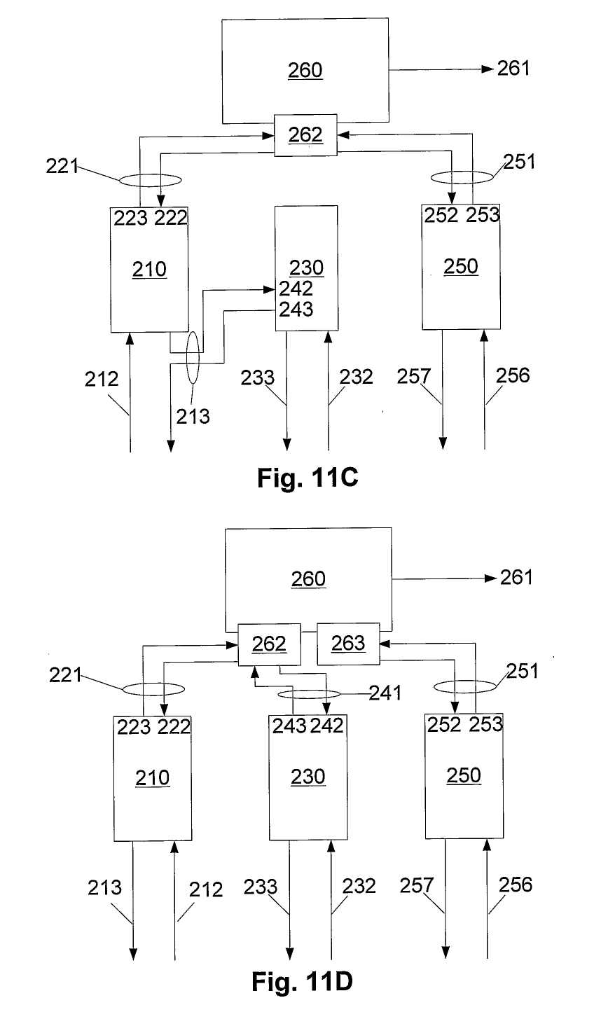

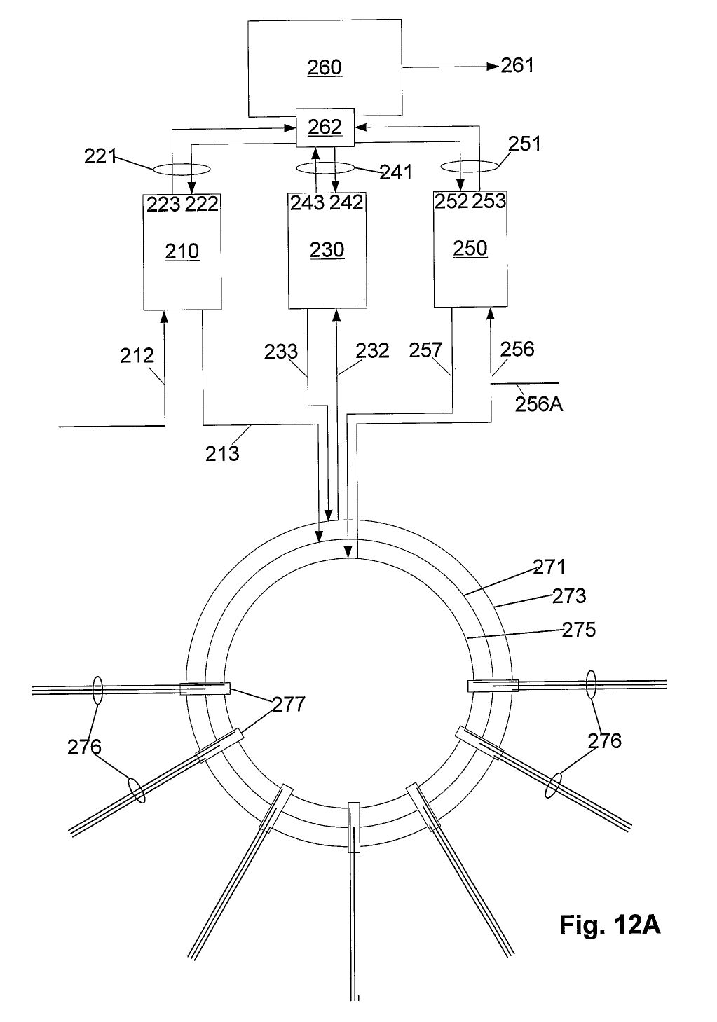

The exact manner in which this is achieved will depend on the respective implementation. Thus, in the example shown in FIG. 10, the heat exchanger 262 is used to heat fluid, such as water, in each of the pipes 221, 241, 251, so that the fluid disinfection system 210, the absorption chiller 230 and the water storage system 250 are each directly heated by waste heat from the generator 260. However alternative interconnections may be used, as shown for example in FIGS. 11A to 11D.

FIG. 11A shows a configuration in which waste heat from the heat exchanger 262 is used to drive the absorption chiller 230. The waste heat 244 generated by the absorption chiller 230 is collected using a heat exchanger, and then used to supply heat to the fluid disinfection system 210 and the water storage system 250, as shown. Other combinations are also possible. For example, the pipes 221, 241, 251 may be connected in parallel as shown in FIG. 10, or in series as shown in FIG. 11B.

Similarly, waste heat from the fluid disinfection system 210, or the water storage system 250 may be used to provide heat to the absorption chiller 230. Thus, for example, the disinfected water provided from the fluid disinfection system 210 via the outlet 213 is typically significantly above ambient temperature, and may therefore be used to provide heat to the absorption chiller 230, as shown in FIG. 11C.

A further variation is shown in FIG. 11D. In this example, the generator is provided with a second heat exchanger 263. In particular, in this instance the heat exchanger 262 will act to remove waste heat from the generator directly, which is usually required in order to maintain the operating temperature of the generator 260. However, additionally waste heat will be present in the exhaust gases from the generator 260, and these can be used to provide independent heat for any one of the fluid disinfection system 210, the absorption chiller 230 and the water storage system 250.

Accordingly, it will be appreciated that the combination of the fluid disinfection system 210, the absorption chiller 230 and the water storage system 250, when coupled to appropriate heat source(s), allows waste heat to disinfect water, generate chilled fluid and provide hot water.

The system therefore allows electricity to be generated in the normal way, and produce hot water, disinfected water and chilled water substantially from waste heat created during the electricity generation. This therefore allows hot and disinfected water, as well as chilled fluid to be provided at substantially no additional operating cost in addition to those incurred producing the electricity.

The chilled fluid can be used to provide air conditioning. This can be achieved for example by circulating the chilled fluid through an appropriate heat exchanger configuration, and allowing air to be blown over the heat exchanger to thereby cool the air. Thus, whilst this would therefore require electricity to drive the fan, and pump the chilled fluid through the heat exchanger, this avoids the need to use an electrically driven compressor to provide air conditioning, thereby further reducing the electrical load required to provide the air conditioning.

As a result, the use of a combination system, such as those described above, vastly reduces the operating and environmental costs involved in providing facilities in resorts or other remote environments, or the like. In fact, the system may be used to generate hot water, chilled fluid and disinfected water using any heat source. This could include for example existing boilers within hospitals, or the like.

An example of a suitable system will now be described with respect to FIGS . 12A and 12B.

In this example, the outlets 213, 233, 257 are connected to a distribution network 270, including three respective distribution pipes 271, 273, 275, as shown. In this example, the pipes are provided in a ring configuration, although this is not essential, with a number of individual distribution branches provided as shown at 276. In general, the distribution pipes 271, 273, 275, would be used for example to circulate hot, disinfected and chilled water throughout a resort, with the branches 276 being used to control distribution to each of a number of buildings.

In order to provide additional control, flow control valves 277, may be used to selectively activate the branches 276, so that fluid supply to each building may be individually controlled.

As the pipes 273, 275 are distributing cold and hot water respectively, it is preferable to ensure that these are adequately insulated. Accordingly, the pipes 271, 273, 275 are typically formed from a material having good inherent insulating properties, such as polypropylene, or the like. The pipes 271, 273, 275 are also typically provided in a trench 280 and positioned using appropriate struts 281, before the trench is filled with insulating foam 282. This not only provides additional insulation, but also helps protect the pipes from damage.

Finally, the pipes are arranged with the pipe 271 disposed between the pipes 273, 275. In this regard, the disinfected water in the pipe 271 is typically at ambient temperature, whilst the pipes 273, 275 contain cold and hot water respectively, and this arrangement therefore minimises heat transfer between the pipes.

In use, the fluid disinfection system will receive water via the inlet 212, disinfect the water, before distributing the disinfected water via the outlet 213, and the respective distribution pipes 271.

The fluid disinfection system described system is suitable for disinfecting a wide range of fluids in high volume. In particular, the system is ideally suited for disinfecting both grey fluid, which includes fluid from showers, wash basins, dishwashers and washing machines, and black fluid, which includes fluid from toilets, septic systems etc, and which typically contains amounts of contaminants, such as Faecal coliforms and bacteria, as well target species of organisms, bacteria, pathogens and compounds, such as oestrogen, nitrates, phosphates, and the like.

It will be appreciated that residual purification, such as filtering or chlorination for residual treatment, may also be provided depending on the attended use of the treated water.

Due to the improved quality of disinfected fluid compared to other fluid cleaning techniques it is possible to utilise the system in a greater variety of circumstances. Thus, for example, the systems can be used to treat effluent from sewage plants and septic tanks, as well as waste water from industry, making it safe for re-use or disposal. In addition to this, animal effluent can be disinfected for use in irrigation of crops or pasture. Polluted river or dam water can be disinfected allowing it to be used as potable water. The system can also be used to disinfect ballast water on boats / ships, allowing the ballast water to be safely returned to the sea or rivers.

When the disinfection system is to treat black fluids, such as sewer water, it is typical to provide preliminary processing of the fluid prior to disinfection. In particular, the black fluid may be provided to a bio digester, including bio filters such as Zabel A300 bio filters. The fluid will then typically undergo further filtering to remove larger pieces of debris, before being disinfected using one of the above described systems, as will be appreciated by persons skilled in the art.

In any event, it will be appreciated that this allows water from a variety of sources to be collected via the inlet 212, and then distributed for a variety of uses, such toilet water, irrigation, cleaning, or the like.

In the case of the absorption chiller, the chilled water is only used for cooling purposes, and this can therefore be recirculated, and will therefore only require occasional replenishment. It will be appreciated from this that in the event that the recirculated water is still below ambient temperature, then the load on the absorption chiller will be reduced.

In any event, as the chilled fluid is generated virtually at no cost, this allows air conditioning to be provided in circumstances that would otherwise be uneconomic. This includes, for example, allowing buildings to be permanently air conditioned, as well as to provide streams of chilled air in external environments, such as around a swimming pool, on a beach, or the like.

In general, the temperature of air produced in this fashion is not as cold as that produced by compression driven air conditioners, but as the air conditioning can be provided permanently at virtually no cost, this allows rooms to be permanently cooled, which assuming sufficient isolation from the environment is provided, will allow a desired room temperature to be achieved regardless of the ambient temperature.

As far as the hot water supply is concerned, it will be appreciated that any unused hot water can be recirculated via the inlet 256 for reheating, with additional water being supplied via the inlet 256A as required.

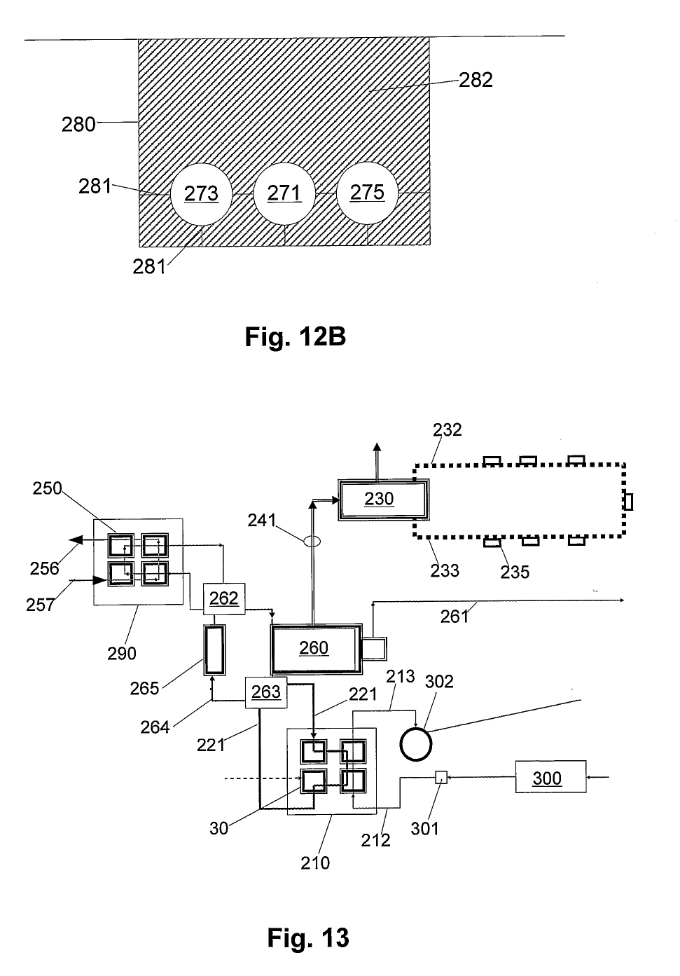

A second example of a system incorporating an absorption chiller, a hot water storage system, a fluid disinfection system, will now be described with reference to FIG. 13.

In particular, as shown a generator 260 is coupled to a fluid disinfection system 210 via a heat exchanger 263 and to a hot water storage system 290 via a heat exchanger 262, which are connected via a pipe 264 as shown. A radiator 265 may also be provided to radiate excess heat if required. As our alternative, the heat exchangers 262, 263 can be removed and the pipe 264 extended to pass directly through the hot water storage system and the disinfection system. However, this can unduly increase the length of pipe 264, which can in turn strain pumps used to pump fluid through the, pipe 264

Additionally exhaust gases are passed along a pipe 241 to an absorption chiller 230 which operates to generate chilled water which is output via the pipe 232 to air-conditioning units 235 as shown. In this particular example, a 500 EPSTP (Estimated People Sewage Treatment Plant) 300 is coupled to a sand filter or participant matter filter 301 to provide water for recycling, via the inlet 212; to the fluid disinfection system 210. Disinfected fluid is output via the outlet 213 to a recycled water storage tank 302. Operation of the remaining element is substantially as described above, although in this example it can be seen that the hot water storage system 290 is formed from a member of Rotex heat storage vessels 250 which are connected in series to provide for heating of water received via the inlet 257 to thereby provide hot water via the outlet 256.

It will be appreciated that the techniques outlined herein could be applied to any form of fluid disinfection system that utilises heat to provide for disinfection or other disinfection of fluid. This could include for example medical applications such as retorting and autoclaving, as well as disinfection systems for the disinfection of milk and the like. It will be appreciated that these applications provide particular benefits.

In particular, in hospitals it is typical to have boilers to supply sufficient hot water for use in washing and the like. In this case, waste heat from the boilers can be used to provide sterilisation of medical equipment, thereby removing the requirement for providing separate sterilisation equipment that uses electric heating of water.

In the case of milk disinfection, in many circumstances the milk is produced on farms that are in remote locations. Due to the inherent delays in transport, it is therefore preferable that the milk is disinfected at source to ensure freshness. However, in such remote situations, electricity is often generated using a generator, and hence using electrically driven disinfection systems places a major load on the generator, and results in heavy fuel use. By utilising the current system, waste heat from the generator (or from the truck motor that is picking the milk up) is employed to disinfect the milk, thereby leading to significant cost savings.

It will be appreciated by persons skilled in the art that disinfection of fluid is commonly referred to as pasteurisation and that accordingly, the above described techniques can equally apply to disinfection and pasteurisation, which is a particular form of pasteurisation.

Persons skilled in the art will appreciate that numerous variations and modifications will become apparent. All such variations and modifications which become apparent to persons skilled in the art, should be considered to fall within the spirit and scope that the invention broadly appearing before described.

1. Apparatus for disinfecting fluid, the apparatus including:
a) a preheat heat exchanger for heating the fluid to a first temperature, the preheat heat exchanger including:
i) a first inlet for receiving the fluid;
ii) a first outlet for supplying preheated fluid at the first temperature;
iii) a second inlet for receiving disinfected fluid substantially at a second temperature; and,
iv) a second outlet for supplying the disinfected fluid; and,
b) a disinfection tank for heating the fluid to a second temperature, the disinfection tank including:
i) a heat source;
ii) an inlet for receiving the preheated fluid;
iii) a heat exchanger coupled to the inlet for heating the preheated fluid to a second temperature to thereby disinfect the fluid; and,
iv) an outlet coupled to the heat exchanger for providing the disinfected fluid to the second inlet of the preheat heat exchanger.

2. Apparatus according to claim 1, wherein the heat exchanger has a predetermined length.

3. Apparatus according to claim 1, wherein the heat exchanger is formed from a convoluted pipe.

4. Apparatus according to claim 1, wherein the heat exchanger is formed from a coiled pipe.

5. Apparatus according to claim 4, wherein the coiled pipe is adapted to reduce the effects of channelling within the pipe.

6. Apparatus according to claim 1, wherein the heat source includes a primary circuit and at least one of:
a) a heating element; and,
b) a second heat exchanger coupled to a source of hot fluid.

7. Apparatus according to claim 6, wherein the hot fluid is heated by at least one:
a) waste heat from equipment; and,
b) solar heating.

8. Apparatus according to claim 1, wherein the disinfection tank is a reverse acting califorier.

9. Apparatus according to claim 1, wherein the disinfection tank is a Rotex™ SC500.

10. Apparatus according to claim 9, wherein the heat exchanger is a PE-X heat exchanger.

11. Apparatus according to claim 1, wherein the preheat heat exchanger is a second reverse acting califorier.

12. Apparatus according to claim 1, wherein the preheat heat exchanger is a second Rotex™ SC500.

13. Apparatus according to claim 12, wherein the second inlet and second outlet are coupled to a primary circuit of the second Rotex™ SC500.

14. Apparatus according to claim 13, wherein the preheat heat exchanger is a PE-X heat exchanger.

15. Apparatus according to claim 1, wherein the disinfection tank includes an insulated housing.

16. Apparatus according to claim 1, wherein the heat source includes pipe coupled to a boiler.

17. Apparatus according to claim 16, wherein the fluid at the second temperature is pressurised.

18. Apparatus according to claim 17, wherein the apparatus includes one or more pressure release valves.

19. Apparatus according to claim 1, wherein the fluid is provided at a predetermined rate, and wherein the heat exchanger is adapted to heat the fluid to the second temperature for a predetermined length of time.

20. Apparatus according to claim 19, wherein the apparatus further includes a control system for controlling the predetermined rate.

21. Apparatus according to claim 20, the control system including:
a) a flow control valve; and,
b) a controller for controlling the flow control valve.

22. Apparatus according to claim 21, wherein the apparatus further includes a temperature sensor which generates signals indicative of the second temperature, and wherein the controller controls the predetermined flow rate in accordance with the signals.

23. Apparatus according to claim 21, wherein the controller is a suitably programmed processing system.

24. A method of operating apparatus for disinfecting fluid, the apparatus including:
a) a preheat heat exchanger for heating the fluid to a first temperature, the preheat heat exchanger including:
i) a first inlet for receiving the fluid;
ii) a first outlet for supplying preheated fluid at the first temperature;
iii) a second inlet for receiving disinfected fluid substantially at a second temperature; and,
iv) a second outlet for supplying the disinfected fluid; and,
b) a disinfection tank for heating the fluid to a second temperature, the disinfection tank including:
i) a heat source;
ii) an inlet for receiving the preheated fluid;
iii) a heat exchanger coupled to the inlet for heating the preheated fluid to a second temperature to thereby disinfect the fluid; and,
iv) an outlet coupled to the heat exchanger for providing the disinfected fluid to the second inlet of the preheat heat exchanger; and,
v) wherein the method includes supplying the fluid to the first inlet at a predetermined rate.

25. (canceled)

26. A supply system including:
a) an absorption chiller for using an external heat source to provide chilled fluid;
b) a fluid disinfection system including a disinfection tank having a heat exchanger extending from an inlet to an outlet for using an external heat source to provide disinfected fluid;
c) a hot water system for using an external heat source to provide heated fluid; and,
d) a waste heat recovery system to recover waste heat, the waste heat recovery system acting as an external heat source for at least one of the absorption chiller, the fluid disinfection system and the hot water storage system.

27. Apparatus according to claim 26, wherein the waste heat recovery system includes a heat exchanger coupled to at least one of:
a) a generator; and,
b) a boiler.

28. Apparatus according to claim 26, wherein the waste heat recovery system provides heat to a selected one of the absorption chiller, the fluid disinfection system and the hot water system, the system further including a second waste heat recovery system for:
a) recovering waste heat from the selected one of the absorption chiller, the fluid disinfection system and the hot water system; and
b) providing the waste heat to one of the absorption chiller, the fluid disinfection system and the hot water system.

29. Apparatus according to claim 26, wherein the absorption chiller includes:
a) an evaporator which uses evaporation of a refrigerant to cool fluid received via an inlet, and provide chilled fluid via an outlet;
b) an absorber for:
i) receiving evaporated refrigerant from the evaporator; and,
ii) causing the evaporated refrigerant to be absorbed by a refrigerant-depleted solution to form a solution;
c) a chiller generator for:
i) receiving the solution from the absorber;
ii) evaporating refrigerant from the solution using an external heat source heat to create the refrigerant-depleted solution; and,
iii) providing the refrigerant-depleted solution to the absorber;
d) a condenser for:
i) receiving evaporated refrigerant from the chiller generator;
ii) condensing the evaporating refrigerant and generating waste heat; and,
iii) providing the refrigerant to the evaporator.

30. Apparatus according to claim 29, wherein the fluid disinfection system includes:
a) a preheat heat exchanger for heating the fluid to a first temperature, the preheat heat exchanger including:
i) a first inlet for receiving the fluid;
ii) a first outlet for supplying preheated fluid at the first temperature;
iii) a second inlet for receiving disinfected fluid substantially at a second temperature; and,
iv) a second outlet for supplying the disinfected fluid; and,
b) a disinfection tank coupled to an external heat source for heating the fluid to a first temperature, the disinfection tank including:
i) an inlet for receiving the preheated fluid;
ii) a heat exchanger coupled to the inlet for heating the preheated fluid to a second temperature to thereby disinfect the fluid; and,
iii) an outlet coupled to the heat exchanger for providing the disinfected fluid to the second inlet of the preheat heat exchanger.

31. Apparatus according to claim 30, wherein the heat exchanger has a predetermined length.

32. Apparatus according to claim 30, wherein the heat exchanger is formed from a convoluted or coiled pipe.

33. Apparatus according to claim 29, wherein at least one of the disinfection tank and the preheat heat exchanger are formed from a reverse acting califorier.

34. Apparatus according to claim 33, wherein the reverse acting califorier is a Rotex™ SC500.

35. Apparatus according to claim 34, wherein the heat exchanger is a PE-X heat exchanger.

36. Apparatus according to claim 26, wherein the hot water supply includes a reverse acting califorier.

37. Apparatus according to claim 36, wherein the reverse acting califorier is a Rotex™ SC500.

38. A supply system including:
a) a fluid disinfection system including a disinfection tank having a heat exchanger extending from an inlet to an outlet for using an external heat source to provide disinfected fluid;
b) a hot water system for using an external heat source to provide heated fluid; and,
c) a waste heat recovery system to recover waste heat, the waste heat recovery system acting as an external heat source for at the fluid disinfection system and the hot water storage system.

39. Apparatus according to claim 38, wherein the waste heat recovery system is coupled to a generator for generating an electricity supply.

40. (canceled)

41. Apparatus for treating ballast water in a vessel, the apparatus including:
a) a preheat heat exchanger for heating the ballast water to a first temperature, the preheat heat exchanger including:
i) a first inlet for receiving the ballast water from a ballast tank;
ii) a first outlet for supplying preheated ballast water at the first temperature;
iii) a second inlet for receiving disinfected ballast water substantially at a second temperature; and,
iv) a second outlet for supplying the disinfected ballast water to the ballast tank; and,
b) a disinfection tank for heating the ballast water to the second temperature, the disinfection tank including:
i) an inlet for receiving the preheated ballast water;
ii) a heat exchanger coupled to the inlet for heating the preheated ballast water to a second temperature to thereby disinfect the ballast water; and,
iii) an outlet coupled to the heat exchanger for providing the disinfected ballast water to the second inlet of the preheat heat exchanger; and,
c) a heat recovery heat system coupled to engines provided in the vessel, the heat recovery system being adapted to heat the disinfection tank, thereby allowing the ballast water to be disinfected.

42. Apparatus according to claim 41, wherein the first inlet is coupled to the ballast tank at a first level and the second outlet is coupled to the ballast tank at a second level, the second level being higher than the first level to thereby ensure disinfected water is returned to the ballast tank at a higher level.

43. (canceled)

44. Apparatus for treating ballast water in a vessel, the apparatus including:
a) a heat recovery heat system for recovering heat from at least one of an engine and a boiler; and,
b) a fluid disinfection system for heating the ballast water to a predetermined temperature using the recovered waste heat, to thereby disinfect the ballast water.

45. (canceled)
